(19) United States
(12) Patent Application Publication
(10) Pub. No.: US 2004/0166120 A1
(43) Pub. Date: Aug. 26, 2004

(54) Vaccination against anthrax

(76) Inventors: Lawrence J. Thomas, South Easton, MA (US); Angelo Scorpio, Boonsboro, MD (US); David T. Beattie, South Natick, MA (US)

Correspondence Address: Leon R Yankwich, Yankwich & Associates, 201 Broadway, Cambridge, MA, 02139, US

(21) Appl. No.: 10/479,770

(22) PCT Filed: Jun. 10, 2002
(86) PCT No.: PCT/US02/18336

(51) Int. Cl. A61K 39/02; A61K 39/07

(52) U.S. Cl. 424/190.1; 424/246.1

(57) ABSTRACT

Methods are disclosed for immunizing a mammal against B. anthracis using a composition of pure recombinant Protective Antigen (rPA), optionally in combination with truncated Lethal Factor polypeptide (LFn). Formulations of the pure rPA immunogen have little or no reactogenicity and therefore may be administered to a mammalian subject in very high doses of 50 μg to 1000 μg or more rPA, which is at least four times the amount of PA included per dose in conventional anthrax vaccines. Preferred immunogenic compositions are free of adjuvant and other undesired components, further enhancing the effectiveness and safety of the compositions. Methods for preparing the immunogenic compositions and for purifying rPA and LFn polypeptides also are disclosed.

### FIELD OF THE INVENTION

The present invention relates to improvements in compositions for eliciting an immune response in a mammal against B. anthracis, methods of administering such compositions to elicit a beneficial immune response, and methods for preparing such compositions.

### BACKGROUND OF THE INVENTION

Anthrax is an infectious bacterial disease caused by Bacillis anthracis. It occurs most commonly in wild and domestic herbivores (sheep, goats, camels, antelope, cattle, etc.) but may also occur in humans. Infection can occur by cutaneous exposure, by ingestion (gastrointestinal anthrax), or by inhalation (pulmonary anthrax). 95% of anthrax infections in humans occur by cutaneous infection, either from contact with unvaccinated, infected animals in an agricultural setting, or by handling contaminated animal products (meat, leather, hides, hair, wool, etc.) in an industrial setting.

Cutaneous anthrax is fatal in about 20% of cases if untreated, but it can usually be overcome with appropriate antimicrobial therapy. Inhalation or gastrointestinal anthrax infection is much more serious and much more difficult to treat. Inhalation anthrax results in repiratory shock and is fatal in 90%-100% of cases; gastrointestinal anthrax results in severe fever, nausea and vomiting, resulting in death in 25%-75% of cases.

An effective vaccine against anthrax was developed in the United States in the 1950s and 1960s, and a vaccine was approved by the FDA in 1970.

In recent years the threat of airborn transmission of anthrax has been thought to increase as B. anthracis was identified as a possible agent for biological warfare. (See, e.g., U.S. Congress, Office of Technology Assessment, Proliferation of Weapons of Mass Destruction: Assessing the Risks, OTA-ISC-559 (Washington, D.C.; U.S. Government Printing Office, August 1993); www.anthrax.osd.mil.) This threat has now been realized in the past year in the form of mailed anthrax spores, resulting in several deaths. Whereas historically only individuals at high risk, such as veterinarians, livestock handlers, wool shearers, abbatoire workers, etc., needed to consider being vaccinated, the threat to military personnel of the possibility of biological weapons deployment caused the United States military to adopt a sweeping anthrax vaccination program in 1997, under which it was intended to administer the anthrax vaccine to 2.4 million military personnel in all branches of service. (See, e.g., Secretary of Defense, Memorandum for Secretaries of the Military Departments et al., May 18, 1998, Implementation of the Anthrax Vaccination Program for the Total Force.)

The only mass produced anthrax vaccine, Anthrax Vaccine Adsorbed (or AVA, commercial name BioThrax™), is a noninfectious sterile filtrate of an attenuated strain of B. anthracis, adsorbed to aluminum hydroxide (alum) adjuvant, with ≦0.02% formaldehyde and 0.0025% benzethonium chloride added. (Friedlander et al., JAMA, 282(22):2104-2106 (1999).) The course of vaccination consists of six subcutaneous injections of 0.5 mL doses of vaccine over eighteen months, with annual boosters to maintain immunity. This vaccination is believed to provide immunity that is 90%-100% effective against aerosol anthrax challenge, based on animal studies and incidental human data. (Friedlander et al., id.)

While the AVA is effective, the vaccine strain employed (i.e., a non-proteolytic, non-capsulated mutant strain of B. anthracis, V770-NP1-R) has some disadvantageous characteristics: Despite its mutations, the strain retains a sporogenic and fully toxogenic phenotype, and use of the whole strain in vaccine production results in lot-to-lot variability in levels of Protective Antigen, as well as inclusion of PA degradation products and other bacterial products, which may include EF and LF. (Farchaus, J., et al., Applied & Environmental Microbiol., 64(3):982-991 (1998).) In addition, perceived side effects from administering the vaccine have recently touched off a major controversy: Prior to the Military's Anthrax Vaccine Immunization Program, or AVIP, the reported side effects ranged from the common injection site swelling and tenderness in 30% of recipients to systemic reactions (malaise, lassitude, fever, chills) in less than 0.02% of recipients. However, by August 1999, AVIP had accounted for administration of over 1,000,000 doses of the vaccine to nearly 350,000 military personnel, and the anthrax vaccine was being accused of causing much more serious side effects, including hair loss, muscle aches, chronic fatigue, aching teeth and gums, thick saliva, burn-like skin reactions, rapid weight loss, blackouts, and at least one death. (See, e.g., Chicago Tribune, Mysterious illnesses strike some gulf vets, Mar. 26, 1992, p.2; The Washington Post, The Nation in Brief, Sep. 29, 2000, Section A, p. 34; www.gulfwarvets.com; www.enter.net/˜jfsorg/.) The suspicion of serious side effects has led to charges that the anthrax vaccine is contaminated, e.g., with squalene (see, Garret, L., Big Battle Over Vaccine: Detractors Say Immunization for Antrhax Hazardous; Pentagon Says No, The Beacon Journal (Akron), Sunday Jul. 4, 1999, Section B, p. 1), and has resulted in hundreds of military personnel refusing to be vaccinated (see, Graham, B., Some in Military Fear Anthrax Inoculation Side Effects, The Plain Dealer (Cleveland), Nov. 26, 1998, Section: National, p. 6E; Air Force Reserve Pilots Quitting Due to Vaccine, The Plain Dealer (Cleveland), Feb. 27, 1999, Section: National, p. 6A). Military personnel ranked as high as major have accepted court-martial and dismissal from military service rather than accept the anthrax vaccine. (Eskenazi, M., How Anthrax Causes Early Retirement, TIME.com, Mar. 31, 2000.)

In view of this background, there is a need for an improved composition for immunization against anthrax that is effective to raise an immune response against B. anthracis but which may be formulated without contaminants that may lead or be suspected of leading to unwanted side effects. Improved methods of administration that avoid the long course of vaccination are also needed. Finally, there is a need for methods of manufacturing and formulating anthrax vaccine compositions to provide ultrapure immunogenic components. These needs are addressed by the present invention, disclosed herein.

### SUMMARY OF THE INVENTION

The present invention provides pure vaccine compositions, having fewer B. anthracis-derived components than the existing approved anthrax vaccine and thus having a reduced risk of side effects.

The present invention provides a composition for raising an anti-B. anthracis antigen immune response in a mammal consisting essentially of recombinant B. anthracis Protective Antigen (rPA). Most preferably, the composition is formulated without the use of adjuvant such as alum.

In an alternative embodiment, a composition is provided for eliciting an anti-B. anthracis immune response in a mammal consisting essentially of recombinant Protective Antigen and a truncated, non-functional (non-toxic) B. anthracis Lethal Factor (LFn). Combination of rPA and LFn components has been found to enhance the anti-LFn antibody titer, in comparison to immunization with LFn alone.

The present invention also provides a method for eliciting an immune response in a mammalian subject against a B. anthracis antigen comprising:

(a) administering to a mammalian subject a composition consisting essentially of recombinant Protective Antigen (rPA),

(b) optionally, repeating said administration one or more times, wherein said administration results in an an anti-PA antibody response in said mammal. Advantageously, the immunogenic compositions according to the invention may be given in high doses (e.g., 50 μg or more) without experiencing the often-observed side effects of prior art anthrax vaccines (e.g., erythema, edema). Also, anti-PA titers exceeding 100 are readily achieved. Particular advantages of the immunization methods of the invention described herein are the ability to employ high doses of immunogen, e.g., greater than 50 μg, up to 1000 μg or more, and the achievement of very high anti-PA antibody titers, e.g., greater than 500, preferably greater than 1000, up to 200,000 or higher.

In preferred features, the present invention provides a method for vaccinating a mammalian subject against B. anthracis infection, which method comprises:

(a) administering to a mammalian subject a composition consisting essentially of recombinant Protective Antigen (rPA),

(b) optionally, repeating said administration one or more times,

wherein said mammalian subject is thereby immunized against B. anthracis infection.

Optionally, the composition administered according to the invention also contains a truncated Lethal Factor potypeptide (LFn), that is, a polypeptide that contains a portion of the B. anthracis Lethal Factor protein but not the full-length protein, particularly a polypeptide lacking the catalytic domain of Lethal Factor. A preferred LFn comprises the N-terminal 254 amino acids of Lethal Factor, or fewer. The use of the two-component composition gives high titers of anti-PA and anti-LF antibodies, and the combination has been discovered to markedly enhance the production of anti-LF antibodies in comparison with LFn administered alone.

Preferably, the immunogenic composition administered according to the method is free of adjuvant. More preferably, the immunogenic composition administered according to the method is free of any B. anthracis proteins naturally associated with Protective Antigen. Most preferably, the immunogenic composition administered according to the method is also free of other proteins and chemicals that have been associated with prior art compositions for obtaining an anti-B. anthracis immune response, such as protease inhibitors, protein inactivators (in particular formaldehyde), chemical preservatives, animal serum or proteins (particularly equine or bovine serum or proteins), and materials prepared from animal serum or proteins.

Preferably, the immunogenic composition administered according to the method is administered in a regimen requiring fewer doses than with the AVA, which follows a regimen of six doses over 18 months. Preferably the method involves administration of an immunogenic composition fewer than six times in a year, most preferably fewer than three times in a year. Alternatively, the dosing regimen may be based on the minimum number of administrations in order to achieve a desired anti-PA antibody titer in an immunized subject. In preferred embodiments, an amount of immunogenic composition sufficient to elicit an antibody titer exceeding 1000 is obtained in three administrations or fewer.

Preferably, the composition is administered in an amount providing at least 50 μg of rPA per dose. This amount is about a four-fold increase in the amount of PA provided in a 0.5 mL dose of the AVA vaccine. The purity of the composition used according to the invention and the absence of additional bacterial and/or adjuvant components as compared to AVA reduces reactogenicity of the instant composition, e.g., decreases the incidence of injection-site reactions (erythema and edema) and other side effects that have become expected with AVA vaccination. For instance, administration of the composition of the present invention to a mammalian subject is accompanied by little or no injection site erythema and swelling, in contrast to at least minor erythema observed in 30% of all vaccinees receiving AVA (Friedlander et al., JAMA, 282(22):2104-2106 (1999)).

Because of the reduced incidence of side effects, the pure rPA compositions of the present invention can be given in much higher doses than AVA without discomfort. For example, an anthrax vaccine composition according to the invention provides at least 50 μg rPA per dose and may advantageously provide, 100 μg rPA per dose, 500 μg rPA per dose, 1000 μg (i.e., 1 mg) rPA per dose or more. Dosages as high as 1000 μg rPA have been administered to test animals according to the invention without significant measurable side effects. Furthermore, it has been surprisingly discovered that high initial doses of rPA lead to very high anti-PA titers that persist over time. The invention therefore provides a new vaccine design for immunization against anthrax infection, utilizing a pure, one- or two-component vaccine, preferably free of adjuvant, in high doses with few repeat administrations (boosts), this in comparision to the AVA, which is a multi-component vaccine obtained from a bacterial filtrate, precipitated on alum, and treated with formaldehyde.

The present invention also provides a method for obtaining high-purity rPA which may advantageously be used for the immunogenic compositions and immunization methods of the present invention. The purification method comprises:

(a) culturing bacterial cells transformed to express recombinant PA,

(b) treating the cells to release the recombinant PA into the culture medium,

(c) purifying the culture medium using a combination of purification steps comprising:

(i) anion exchange chromatography,

(ii) hydroxyapatite chromatography,

(iii) hydrophobic interaction chromatography,

(iv) size exclusion chromatography.

In the foregoing method, preferably the host bacterial cells are E. coli cells transformed to produce rPA. Also, preferably, the hydroxyapatite chromatography step utilizes a ceramic hydroxyapatite matrix. Preferably the chromatography steps are performed in the same order depicted above (i through iv).

The present invention also provides a method for obtaining high-purity recombinant LFn polypeptides which may advantageously be used for the immunogenic compositions and immunization methods of the present invention. The purification method comprises:

(a) culturing bacterial cells transformed to express recombinant LFn,

(b) lysing the cells,

(c) purifying the cell lysate using a combination of purification steps comprising:

(i) immobilized metal affinity chromatography,

(ii) anion exchange chromatography,

(iii) hydrophobic interaction chromatography,

(iv) size exclusion chromatography.

In the foregoing method, preferably the host bacterial cells are E. coli cells transformed to produce rLFn. Preferably the chromatography steps are performed in the same order depicted above (i through iv).

### BRIEF DESCRIPTION OF THE DRAWINGS

FIG. 1 is a chart showing scores for injection site erythema in three groups of three New Zealand White rabbits given a series of three injections of 50 μg rPA, 56 μg LFn, or 50 μg rPA and 56 μg LFn together in saline (no adjuvant) intramuscularly. Observations were made after initial vaccination (day 1) and after each of two booster injections (at days 15 and 29). Injections were made in the thigh muscle, alternating sides for each injection. The injection sites were observed for seven days after each injection and erythema scored on a scale of 0-4 (see scoring scale, Table 2, infra).

FIG. 2 is a chart showing scores for injection site swelling in three groups of three New Zealand White rabbits given a series of three injections of 50 μg rPA, 56 fig LFn, or 50 μg rPA and 56 μg LFn together in saline (no adjuvant) intramuscularly. Observations were made after initial vaccination (day 1) and after each of two booster injections (at days 15 and 29). Injections were made in the thigh muscle, alternating sides for each injection. The injection sites were observed for seven days after each injection and swelling scored on a scale of 0-4 (see scoring scale, Table 2, infra).

FIG. 3 is a graph showing mean anti-PA antibody titers from New Zealand White rabbits administered rPA or rPA+LFn and the persistence of antibody titer over time (>224 days).

FIG. 4 is a graph showing mean anti-LFn antibody titers from New Zealand White rabbits administered LFn or rPA+LFn and the persistence of antibody titer over time (>224 days).

FIG. 5 is a series of bar graphs showing anti-OspA antibody titers measured after immunizing BALB/c mice with either an LFn-OspA fusion protein or an LFn-OspA fusion protein in combination with rPA.

### DEFINITIONS

In order that the invention may be clearly understood, the following terms are defined:

The term “recombinant” is used herein to describe non-naturally altered or manipulated nucleic acids, host cells transfected with exogenous nucleic acids, or polypeptide molecules that are expressed non-naturally, through manipulation of an isolated nucleic acid (typically, DNA) and transformation or transfection of host cells. “Recombinant” is a term that specifically encompasses nucleic acid molecules that have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides.

The terms “recombinant Protective Antigen”, “rPA”, and “recombinant PA” as used herein all refer to a recombinantly produced polypeptide having the functional activity of the native Protective Antigen protein of Bacillus anthracis, Mᵣ85,000 and pI 5.5, which is one component of the anthrax binary toxin. That is, the rPA combined with B. anthracis Lethal Factor provides a toxin lethal to cells (i.e., macrophages) or laboratory animals (e.g., rats). Recombinant rPA is also defined, alternatively, as a polypeptide produced according to recombinant DNA techniques and having the ability to elicit an antibody response in mammals such as rabbits or mice, which antibodies are immunologically cross-reactive with natural B. anthracis Protective Antigen. The gene for Protective Antigen has been cloned and sequenced. (See, Vodkin, M., et al., Cell, 34:693 (1983); Welkos, S., et al., Gene, 69(2):287-300 (1988).)

The terms “Lethal Factor fragment”, “truncated Lethal Factor”, and “LFn” as used herein all refer to a synthetically or recombinantly produced polypeptide essentially identical to a non-toxin-forming, N-terminal portion of the native Lethal Factor protein of Bacillus anthracis, Mᵣ 87,000 and pI 5.8, which is another component of the lethal anthrax binary toxin. An example of an LFn polypeptide according to this definition is a polypeptide consisting of amino acids 1 to 254 of native B. anthracis Lethal Factor. Such a polypeptide includes the PA-binding functionality of the native protein but does not form a lethal toxin when combined with full-length PA. The lethal toxin forming activity of the 776-amino acid Lethal Factor protein is eliminated by removal of the C-terminal 47 amino acids; therefore, a suitable LFn for purposes described herein is a polypeptide consisting of up to the N-terminal 729 amino acids of Lethal Factor. Lethal Factor has been cloned and sequenced. (See, Robertson, D., et al., Gene, 44:71 (1986); Bragg, T., et al., Gene, 81(1):45-54 (1989).) In the present invention, the LFn fragment may be fused to another protein fragment, especially a heterologous antigen (such as, for example, OspA) for introduction of the fusion partner into a target cell, according to methods described in WO 94/18332, WO 97/23236, and WO 98/11914, incorporated herein by reference.

The term “polypeptide”, as used herein, refers to a linear polymer of two or more amino acid residues linked with a peptide bond. Thus, the term “polypeptide” is not restricted to any particular upper limit of amino acid residues.

### DETAILED DESCRIPTION

Bacillus anthracis secretes three proteins which collectively are known as anthrax toxin, Protective Antigen (PA, 85 kD), Lethal Factor (LF, 87 kD), and Edema Factor (EF, 89 kD). None of the proteins individually is toxic, rather the PA protein combines with either LF or EF to form one of two binary toxins. PA and LF together form a lethal toxin; PA and EF together form a toxin that causes edema. (See, e.g., Leppla, Methods in Enzymology, 165:103-116 (1988).) The virulence of wild-type B. anthracis depends on the production of two materials, anthrax toxin (PA, LF and EF) and a polyglutamic acid capsule. These materials are located on separate plasmids in virulent B. anthracis strains, pXO1 (encoding the toxin) and pXO2 (encoding the polyglutamic acid capsule). B. anthracis strains can be made less virulent by eliminating either or both plasmids, and a pXO1⁺ and pXO2⁻ strain was isolated by M. Sterne which was 10⁵ times less virulent than wild-type. (Hainbleton, P., et al., Vaccine, 2:125 (1984).) Such pX01⁺/pXO2⁻ strains are now known as “Sterne-type” strains. A Sterne-type strain selected by the Michigan Department of Public Health for preparation of the human vaccine (AVA) was a non-proteolytic, non-capsulated mutant of B. anthracis, V770-NP1-R (ATCC accession no. 14185). The licensed anthrax vaccine (AVA) is produced by growing the pX01⁺/pXO2⁻ strain in minimal medium in the presence of bicarbonate under microaerophilic conditions and adsorbing the sterile filtered culture supernatant to aluminum oxyhydroxide adjuvant. (See, e.g., Farchaus, J., et al., Applied & Environmental Microbiol., 64(3):982-991 (1988)) and references cited therein. Formaldehyde, in a final concentration not to exceed 0.02% and 0.0025% benzethonium chloride are added to the mass produced vaccine. (AVA Product Insert, BioPort Corporation (www.bioport.com), March 1999.)

In view of the production variability and inclusion of possible unwanted components in the filtered and alum-adsorbed AVA, we sought a simpler vaccine design which would elicit an adequate immune response while eliminating undesired or ineffective components. The present invention is based on the observations that highly pure recombinant Protective Antigen (rPA) may be administered to a mammalian subject to elicit a strong immune response, that the rPA may be administered in much higher doses than contemplated in the prior art without adverse side effects, that high antibody titers following immunization may be achieved without the use of adjuvant, and that rPA itself operates to have an adjuvant effect on optional additional components in an immunogenic composition, in particular LFn, resulting in the production in a subject of higher levels of anti-LF antibodies than observed after immunization using LFn alone. The culmination of these surprising observations leads to the simplified immunogenic composition consisting essentially of rPA and optionally, in addition, LFn claimed herein, and to the use of such compositions to elicit an immune response in mammalian subjects.

The critical ingredient in the immunogenic compositions according to the invention is recombinant Protective Antigen. As mentioned previously, the gene for native PA has been isolated and the sequence published. (See, Vodkin, M., et al., Cell, 34:693 (1983); Welkos, S., et al., Gene, 69(2):287-300 (1988).)

An optional second component of the immunogenic compositions according to the present invention is LFn, which may be any N-terminal fragment of the B. anthracis Lethal Factor capable of eliciting anti-LF antibodies and incapable of forming the lethal binary toxin when administered in concert with rPA. Letaal Factor has also been cloned and sequenced. (See, Robertson, D., et al., Gene, 44:71 (1986); Bragg, T., et al., Gene, 81(1):45-54 (1989).) Preferred LFn polypeptides comprise the N-terminal portion of Lethal Factor necessary to bind to Protective Antigen but does not include the catalytic domain of Lethal Factor. Most preferably, LFn consists essentially of amino acids 1-254 of native Lethal Factor. The 254-amino acid LFn contains the PA-binding domain of LF but not the catalytic domain necessary to form anthrax toxin. Moreover, LFn that includes the PA-binding domain will be useful for introducing an LFn fusion partner, e.g., a subunit vaccine, into target cells, according to methods described in WO 94/18332, WO 97/23236, and WO 98/11914.

Recombinant PA and LFn may be produced using recombinant DNA techniques, utilizing nucleic acids (polynucleotides) encoding the PA or LFn polypeptides and expressing them recombinantly, i.e., by manipulating host cells by introduction of exogenous nucleic acid molecules in known ways to cause such host cells to produce the desired rPA and rLFn.

The polynucleotides coding for PA or LFn may be in the form of RNA or in the form of DNA, which DNA includes cDNA and synthetic DNA. The coding sequences for PA and LFn polypeptides for use according to the present invention may be manipulated or varied in known ways to yield alternative coding sequences that, as a result of the degeneracy of the genetic code, encode the same polypeptide.

Where recombinant production of PA or LFn polypeptide is desired, the present invention also contemplates vectors that include polynucleotides encoding PA or LFn, host cells that are genetically engineered with such vectors, and recombinant polypeptides produced by culturing such genetically engineered host cells. Host cells are genetically engineered (transduced or transformed or transfected) with the vectors, which may be, for example, cloning vectors or expression vectors. The vector may be, for example, in the form of a plasmid, a viral particle, a phage, etc. The engineered host cells can be cultured in conventional nutrient media modified as appropriate for activating promoters, selecting transformants or amplifying the PA- or LFn-encoding polynucleotides. The culture conditions, such as temperature, pH and the like, are those suitable for use with the host cell selected for expression and will be apparent to the skilled practitioner in this field. The polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the selected host. The appropriate DNA sequence may be inserted into the vector by a variety of procedures. In general, the DNA sequence is inserted into an appropriate restriction endonuclease site(s) by procedures known in the art. Such procedures and others are within the capability of those skilled in the art.

The DNA sequence in the expression vector is operatively linked to an appropriate expression control sequence(s) (promoter) to direct mRNA synthesis. As representative examples of such promoters, there may be mentioned LTR or SV40 promoter, the E. coli. lac or trp, the phage lambda PL promoter and other promoters known to control expression of genes in prokaryotic or eukaryotic cells or their viruses. The expression vector also contains a ribosome binding site for translation initiation and a transcription terminator. The vector may also include appropriate sequences for amplifying expression. In addition, expression vectors preferably will contain one or more selectable marker genes to provide a phenotypic trait for selection of transformed host cells, such as dihydrofolate reductase or neomycin resistance for eukaryotic cell culture, or such as tetracycline or ampicillin resistance for bacterial cell cultures such as E. coli.

The vector containing the appropriate DNA sequence as hereinabove described, as well as an appropriate promoter or expression control sequence, may be employed to transform an appropriate host to permit the host to express the protein. As representative examples of appropriate host cells, there may be mentioned bacterial cells, such as E. coli, Streptomyces, Salmonella typhimurium; fungal cells, such as yeast; insect cells such as Drosophila and Sf9; animal cells such as CHO, COS or Bowes melanoma; plant cells, etc. The selection of an appropriate host for this type of recombinant polypeptide production is also within the capability of those skilled in the art from the teachings herein. Many suitable vectors and promoters useful in expression of PA and LFn are known to those of skill in the art, and many are commercially available. The following vectors are provided by way of example. Bacterial: pQE70, pQE60, pQE-9 (Qiagen), pbs, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pWLNEO, pSV2CAT, pOG44, pXTI, pSG (Stratagene) pSVK3, pBPV, pMSG, pSVL (Pharmacia). Any other plasmid or vector may be used as long as it is replicable and viable in the selected host cell.

Introduction of the vectors into the host cell can be effected by any known method, including calcium phosphate transfection, DEAE-Dextran mediated transfection, or electroporation (see Davis et al., Basic Methods in Molecular Biology, (1986)).

One of the principal objects of the present invention is the preparation of immunogenic compositions based on a PA protein that are so pure as to exclude other B. anthracis proteins with which PA is normally associated, in particular full-length Lethal Factor and Edema Factor but also other B. anthracis proteins, i.e., especially bacterial molecules that might be associated with side effects or suspected of causing adverse reactions in vaccinated subjects (e.g., bacterial lipids, lipopolysaccharide molecules, etc.). For this reason, it is most preferred that the PA component of the immunogenic composition of the invention is a recombinant PA and not a natural PA isolated by purification steps from a virulent or avirulent strain of B. anthracis. Similar concerns apply to the optional LFn component, and it is therefore most preferred that the LFn used according to this invention is recombinant LFn (rLFn), although highly purified sythetically produced LFn polypeptides may also be used. Preferably, the PA and LFn polypeptides used according to this invention are also free of other proteins and chemicals that have been associated with prior art compositions for obtaining an anti-B. anthracis immune response, such as protease inhibitors, protein inactivators, chemical preservatives (in particular formaldehyde), and animal serum proteins (particularly equine or bovine serum proteins).

For production of rPA and rLFn, it is most preferred to use E. coli as a host. The use of a bacterial signal sequence, such as that for the E.coli outer membrane protein A (OmpA), is also preferred. Suitable vectors for E. coli production of rPA are familiar to those skilled in the art. See, e.g., Sharma, M., et al., Prot. Expr. & Purif, 7:33-38 (1996).

It is most preferred that the PA and optional LFn components of the immunogenic compositions according to the invention are pure, meaning that the PA or LFn polypeptides have been isolated and purified to substantial homogeneity. A polypeptide that produces a single peak that is at least 95% of the input material on an HPLC column is considered “pure” for the purposes of this invention. For example, rPA or LFn analytically separated as a single peak that is at least 95% of input on a reversed-phase high performance liquid chromatography (RP-HPLC) column, such as a Poros R1/20 column, using a 2-propanol/water gradient is “pure” for the purposes described herein. Preferably, the rPA or LFn component of the compositions disclosed herein will be a polypeptide that produces a single peak that is at least 95%, more preferably at least 97%, more preferably at least 98%, more preferably at least 99% and even more preferably 99.5% or more of the input material on an HPLC column. Utilizing proteins of high purity is believed to contribute directly to the advantageous features of the present invention: i.e., the use of very high amounts of rPA per dose, the preferable absence of adjuvant materials such as alum, and the preferable elimination of common contaminants or additives used in prior art anthrax vaccines. These features, in turn, are believed to contribute to the lack of injection-site erythema and swelling when utilizing compositions according to this invention. At least minor erythema and swelling are common side effects with the AVA (see, Friedlander et al., JAMA, 282(22):2104-2106 (1999)).

Any known method of purification that is suitable for producing pure rPA or LFn polypeptides may be used. A novel multi-step purification method for isolating rPA from bacterial cell culture has been devised that produces pure polypeptides suitable for use according to the present invention. This method involves the use of four chromatographic steps: (i) anion exchange chromatography, (ii) hydroxyapatite chromatography, (iii) hydrophobic interaction chromatography, (iv) size exclusion chromatography, preferably, but not critically, performed in that order. A novel multi-step purification method for isolating rLFn from bacterial cell culture has been devised that produces pure polypeptides suitable for use according to the present invention. This method involves the use of four chromatographic steps: (i) immobilized metal affinity chromatography, (ii) anion exchange chromatography, (iii) hydrophobic interaction chromatography, (iv) size exclusion chromatography, preferably, but not critically, performed in that order. Suitable materials for performing each of these chromatographic steps are known to those skilled in the art.

Preferred immunogenic compositions according to the invention are formulations of rPA providing at least 50 μg per dose of rPA. The AVA composition now in use provides a 0.5 mL dose containing about 10-12 μg PA. Therefore, the preferred compositions of the present invention provide at least a four-fold increase per dose in the amount of PA administered. Because of the absence of side effects observed using compositions according to the invention, much higher doses of rPA may be used, e.g., an immunogenic composition according to this invention may provide 100 μg, 250 μg, 500 μg, 750 μg, 1000 μg (i.e., 1 mg) or more of rPA per dose. The use of high doses has been discovered to lead to enhanced immune responses, as measured by resultant anti-PA antibody titers in immunized subjects, and accordingly the method of vaccination of the present invention preferably comprises administration of fewer doses of rPA in order to obtain a desired level of anti-PA immune activity. Preferably, a desired anti-PA antibody titer will be obtained in a subject with fewer doses of the immunogenic composition than the regimen employed with AVA: six doses administered over 18 months. More preferably, the method of the present invention involves administration of four doses or fewer to obtain an anti-PA antibody titer in an immunized mammalian subject such as a human exceeding 100. More preferably, an antibody titer of 1000 or more is achieved with administration of four doses or fewer. Even more preferably, an antibody titer if 1000 or more is achieved with administration of three doses or fewer. Most preferably, protective immunity to B. anthracis is imparted to the immunized subject.

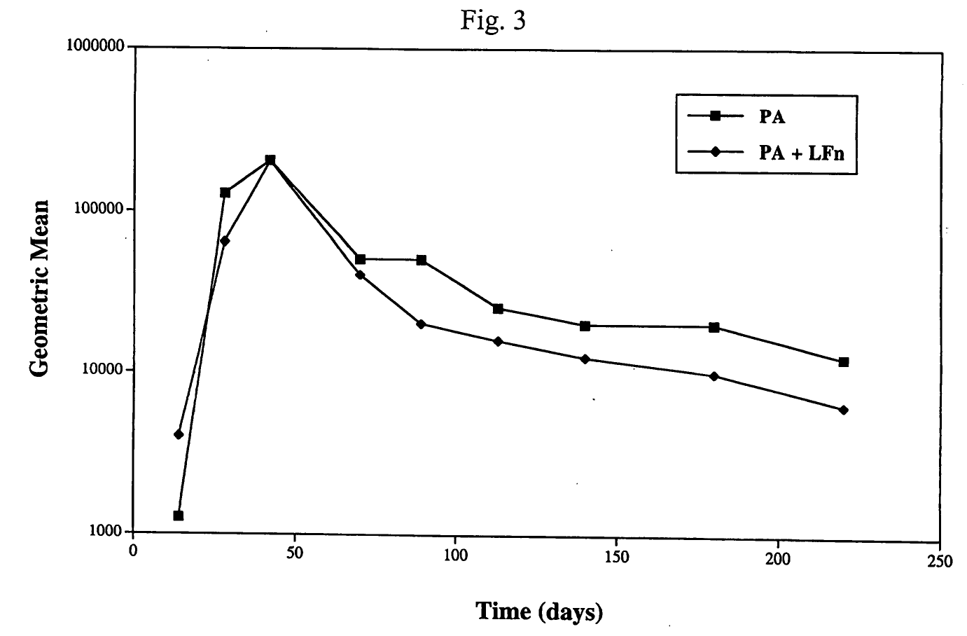

Anti-PA titer, measured as the reciprocal of the dilution of serum at which no PA-reactive antibody is detected, is a common measure of the effectiveness of anthrax vaccines. (See, e.g., Pittman et al., Vaccine, 19:213-216 (2000)), investigating anti-PA titers after two injections in human subjects receiving AVA, where achieving an anti-PA titer of 100 after two injections was considered significant. The method of immunization described herein involves administering an initial dose of an rPA composition, optionally followed by repeated administrations, or boosts, over time. The interval between repeated administrations of the immunogenic composition may vary, and judicious spacing of the doses can increase the immune response, as measured by anti-PA titer. (Pittran et al., id.) Any spacing of doses may be employed that achieves the desired immune response. Administration of immunogenic rPA compositions of the invention according to the methods of the invention preferably results in anti-PA antibody titers of greater than 1000, more preferably greater than 5000, more preferably greater than 10,000, more preferably greater than 50,000, more preferably greater than 100,000 or higher. Mean anti-PA titers as high as about 200,000 have been achieved in mammalian subjects using the compositions and methods of the invention in a series of three administrations of 0.5 mL doses of 50 μg rPA in saline (see, FIG. 3).

Preferably, the immunogenic compositions of the present invention are also prepared without adjuvants. It has been found that rPA may be administered in high doses to mammalian subjects without adjuvant and still elicit a very high titer of anti-PA antibodies. In particular, it is most preferred that compositions administered according to the method of the invention are free of aluminum-based adjuvants variously known as “alum”, e.g., aluminum hydroxide, aluminum oxyhydroxide, aluminum phosphate, etc.

The immunogenic compositions of the present invention may be formulated by dispersing rPA in the desired amount in any pharmaceutical carrier suitable for use in vaccines. Typical doses of anthrax vaccine are 0.5 mL in volume, but any volume suitable to deliver the desired amount of rPA can be used, for example, 0.05 mL to 1.0 mL or more. Accordingly, a typical immunogenic composition according to the invention may be a solution of rPA dispersed in a pharmaceutical carrier providing 50 - 1000 or more μg rPA per 0.5 mL of solution. Any pharmaceutical carrier suitable for administration to mammals which does not interfere with the immunogenicity of the rPA may be employed. Preferred carriers are sterile “water for injection”, saline, and Ringer's Solution.

In view of the discoveries herein, a preferred embodiment of the present invention is a vaccination kit comprising one or more containers of at least 50 μg rPA in a formulation for injection (iv, intramuscular, subcutaneous or intraperitoneal, preferably iv) together with instructions for following the vaccination method of the present invention. Advantageously, the kit could contain, e.g., three or four sterile ampules, each ampule containing one dose of 50 -1000 or more μg of rPA (and optionally 50 - 1000 μg or more of LFn polypeptide in addition), such ampules representing a vaccination regimen of an initial immunization plus one, two or three booster injections.

An optional additional immunogenic component in the compositions of the invention is an LFn polypeptide. Such polypeptides are included to elicit production of antibodies recognizing anthrax Lethal Factor in addition to the anti-Protective Antigen immune response elicited by the rPA component of the composition. Any amount of LFn suitable for eliciting the production of anti-LF antibodies in the immunized subject may be used. Preferably, at least 50 μg LFn per dose will be included in the composition.

Compositions of the invention may be administered to any mammal including humans in which it is desired to elicit an immune response against B. anthracis. In addition to humans, the compositions of the present invention may advantageously be administered, for example, to horses, cattle, oxen, goats, sheep, dogs, cats, antelope, buffalo, rabbits, pigs, and the like.

Compositions of the invention may be administered in any manner used for administration of vaccines. Preferably, the compositions according to the invention will be administered subcutaneously, intradermally, intramuscularly, intravenously, or orally. The most preferred means of administration is via subcutaneous or intramuscular injection.

The following examples are provided to further illustrate the compositions and methods of the present invention. They are provided for illustration and not for limitation of the invention.

### EXAMPLE I

Recombinant PA was produced in E. coli from the strain, E. coli BL21 (DE3)/pET-26bPA, which was prepared by inserting a PA structural gene in a commercially available plasmid, pET-26b, suitable for expression of heterologous proteins in E. coli (Novagen; Madison, Wis.). The pET-26bPA expression vector includes genomic DNA encoding PA linked to the E. coli OmpA secretion signal, under the control of the lacZ inducible promoter, with a kanamycin resistance marker. The transformed E. coli cells were used to seed starter cultures to serve as the inoculum for two 10 liter batch cultures. Following IPTG induction of rPA expression during the fermentation run, a crude preparation of rPA was made by periplasmic release (osmotic shock), nuclease treatment, concentration and filtration. Following these steps, rPA was purified from the crude extract using four column chromatography steps: anion exchange, ceramic hydroxyapatite, hydrophobic interaction, and gel filtration.

The chromatography was all performed on an AKTA FPLC chromatography workstation (Amersham Pharmacia; Uppsala SE), with the control and data collection done using its associated computer running the Unicorn automation and data management software package. Anion exchange chromatography was performed using 137 mL of Q Sepharose HP resin in an XK50/20 column (diameter 5.0 cm, bed height 7.0 cm). Periplasmic protein was loaded onto the column in 20 mM triethanolamine buffer, pH 8.0. After the sample was loaded, the column was washed with 300 ml of 20 mM triethanolamine buffer. Proteins were eluted from the column with a linear gradient of NaCl. The gradient was from 100% 20 mM triethanolamine buffer to 80% 20 mM triethanolamine buffer, followed with a wash of 20% 20 mM triethanolamine/2M NaCl buffer over 7 column volumes (1050 mL) at 10 ml per minute. Fractions (10 mL) were collected throughout the gradient.

Fractions containing rPA were loaded onto a ceramic hydroxyapatite (CHT) chromotography column (diameter 5.0 cm, bed height 7.0 cm). The column was washed with 300 mL 100 mnM sodium phosphate, pH 6.8. Proteins were eluted from the column with a linear phosphate/pH gradient. The gradient was from 100% 10 mM sodium phosphate buffer, pH 6.8, to 100% 400 mM sodium phosphate buffer, pH 8.8, over 500 mL at 10 mL per minute. Fractions (10 mL) were collected throughout the gradient.

Fractions containing rPA were loaded onto a hydrophobic interaction chromatography column (diameter 5.0 cm, bed height 8.0 cm) in 25 mM sodium phosphate/1 M ammonium sulfate, pH 8.0. The column was washed with the same buffer, and the proteins were eluted with a linear gradient of decreasing ammonium sulfate. The gradient was from 100% 25 mM sodium phosphate/1M ammonium sulfate buffer to 100% 25 mM sodium phosphate buffer over 500 mL at 10 mL per minute. Fractions (10 mL) were collected throughout the gradient.

Fractions containing rPA were loaded onto a Sephadex G-15 resin gel filtration column (bed height 48 cm), and the column was washed with 25 mM sodium phosphate buffer. Proteins were eluted with buffer, and fractions collected.

A recombinant LFn polypeptide comprising amino acids 1-254 of Lethal Factor was produced in E. coli from the strain, E. coli BL21 (DE3)/pET-15bLFn, which was prepared by inserting an LFn structural gene into a commercially available plasmid, pET-26b, suitable for expression of heterologous proteins in E. coli (Novagen; Madison, Wis.). The pET-15bLFn expression vector includes genomic DNA encoding the N-terminal 254 amino acids of LF linked to the E. coli OmpA secretion signal, under the control of the lacZ inducible promoter, with a kanamycin resistance marker. Following IPTG induction of rLFn expression, a crude preparation of rLFn was made by homogenization, nuclease treatment, concentration and filtration. The recombinant LFn polypeptide was purified by a combination of metal affinity, anion exchange, hydrophobic interaction and gel filtration chromatography.

Immobilized metal affinity chromatography (IMAC) was performed using 156 mL Chelating Sepharose HP resin (Amersham Pharmacia) in an XK50/20 column (diameter 5.0 cm, bed height 7.8 cm). Whole cell lysate containing LFn was loaded onto the column in 100 mM triethanolamine/0.1 M NaCl. After the sample was loaded, the column was washed with 300 mL 20 mM triethanolamine/0.1 M NaCl buffer and loosely bound proteins were removed from the column by washing with 300 mL wash buffer (100 mM triethanolamine, 60 mM imidazole, 500 mM NaCl, pH 7.9). Proteins were eluted from the column by an increase in imidazole concentration. This was done by stepping from 100% wash buffer to 60% wash buffer and 40% elution buffer (100 mM triethanolamine, 500 mM imidazole, 500 mM NaCl, pH 7.9). Fractions (100 mL) were collected throughout the step, and the fractions containing a large peak of absorbance at 280 nm were removed and stored overnight at −20° C.

Fractions containing LFn were loaded onto an anion exchange chromatography columns (176 mL Q Sepharose HP resin in an XK50/20 column, diameter 5.0 cm, bed height 9.0 cm). After the sample was loaded, the column was washed with 300 mL 20 mM triethanolamine buffer. Proteins were eluted from the column with a linear gradient of NaCl from 0 M to 2 M NaCl. Fractions (10 mL) were collected throughout the gradient.

Fractions containing LFn were loaded onto a hydrophobic interaction chromatography column (diameter 5.0 cm, bed height 10.5 cm). Samples were prepared for loading by adding 0.5 volumes of 20 mM triethanolamine/4 M ammonium sulfate, pH 8.0, buffer to achieve a final concentration of 1.33 M ammonium sulfate. When the sample was completely loaded, the column was washed with 400 mL of 20 mM triethanolamine/1.33 M ammonium sulfate, pH 8.0. Proteins were eluted from the column with a linear gradient of decreasing ammonium sulfate. The gradient was from 100% 20 mM triethanolamine/1.33 M ammonium sulfate buffer to 100% 20 mM triethanolamine buffer over 100 mL at 10 mL per minute. Fractions (10 mL) were collected throughout the gradient.

Samples eluted from the anion exchange column containing LFn were loaded on a gel filtration chromatography column (400 ml Sephadex G-15 resin in an XK50/30 column, bed height 20 cm). Samples were loaded in 25 mM sodium phosphate/150 mM NaCl buffer, and the column was washed with 25 mM sodium phosphate/150 mM NaCl buffer. Fractions containing LFn were collected.

Both recombinant proteins (rPA and rLFn) were obtained at >95% homogeneity.

### EXAMPLE II

Immunogenic compositions were formulated by dispersing the desired amount of rPA or LFn, or combinations thereof, in sterile saline. Dosage volumes were 0.5 mL. Three groups of three male New Zealand White rabbits (1.5-2 kg each, from Millbrook Breeding Labs, Amherst, Mass.) were administered one of three immunogenic compositions in a series of four intramuscular (i.m.) injections. The injections were in alternating thigh muscles. The initial set of three injections (initial vaccination plus two boosts) were administered approximately two weeks apart (specifically, on day 1, day 15, and day 29). The fourth injection was administered over a year later, in Week 78 after the start of the trial. The parameters of the immunization are outlined in Table 1, below:

TABLE 1
Immunization with rPA, LFn, or LFn + rPA

| Compo- | rPA | rLFn | Dose | Injections | Blood Sample Taken |
|---|---|---|---|---|---|
| sition | Dose | Dose | vol. | (Week) | (Week) |
| LFn + | 50 μg | 56 μg | 0.5 mL | 1, 3, 5, 78 | 1*, 3, 5, 7, 11, 14, 17, |
| rPA | | | | | 21, 27, 33, 38, 45, 49, |
| | | | | | 53, 63, 74, 78, 82, 89 |
| LFn | — | 56 μg | 0.5 mL | 1, 3, 5, 78 | 1*, 3, 5, 7, 11, 14, 17, |
| only | | | | | 21, 27, 33, 38, 45, 49, |
| | | | | | 53, 63, 74, 78, 82, 89 |
| rPA | 50 μg | — | 0.5 mL | 1, 3, 5, 78 | 1*, 3, 5, 7, 11, 14, 17, |
| only | | | | | 21, 27, 33, 38, 45, 49, |
| | | | | | 53, 63, 74, 78, 82, 89 |

*blood sample taken prior to initial injection

Animals were monitored daily for feed and water consumption and distress. Rabbits were weighed before each blood sample was taken. Reactogenicity of the immunogenic compositions was monitored by observing injection sites once a day for seven days following each injection. The injection site observations were recorded using a prevalent scoring system for monitoring reactogenicity of injectable vaccines such as AVA. Redness (erythema) and swelling were separately scored using the five-point scales as set forth in Table 2:

TABLE 2
Injection Site Scoring

| Score | Grade | Erythema | Swelling |
|---|---|---|---|
| 0 | none | normal skin color | no swelling |
| 1 | minimal | light pink; indistict | slight swelling; indistinct |
| | | | border |
| 2 | mild | bright pink or pale red; | defined swelling; distinct |
| | | distinct | border |
| 3 | moderate | bright red | defined swelling; raised |
| | | | border (˜1 mm) |
| 4 | severe | dark red; pronounced | pronounced swelling; raised |
| | | | border (>1 mm) |

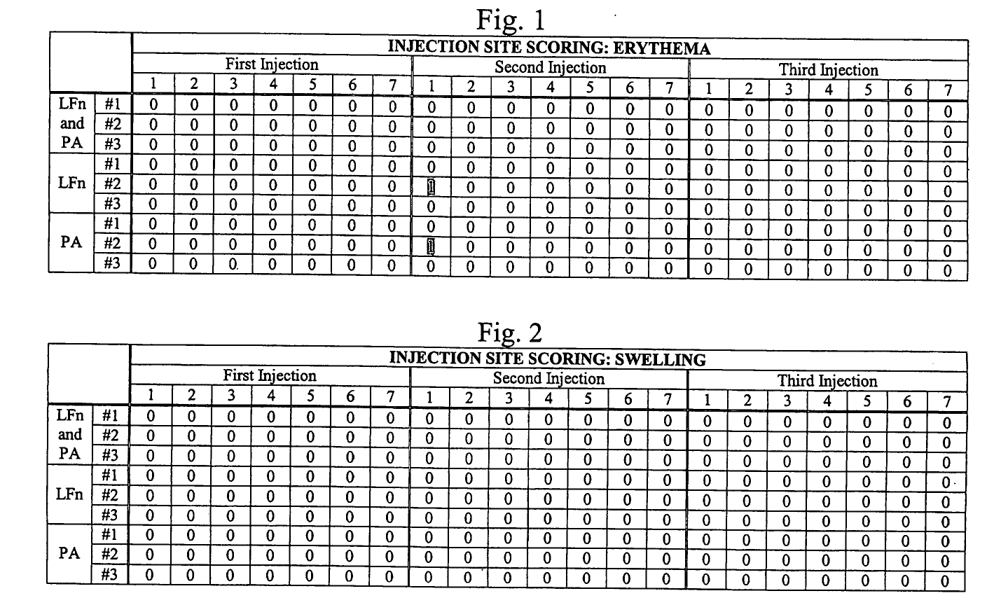

The results of injection site scoring for all three groups are presented in FIG. 1 (erythema) and FIG. 2 (swelling). In all of the observations, only two instances of “minimal” reaction were observed (FIG. 1). All other injections showed zero scores (no reaction).

### EXAMPLE III

Immunogenicity of the compositions was measured using anti-PA and anti-LF ELISAs.

Microtiter plates (from PGC Scientific, Gaithersburg, Md., cat. #5-6114-06) were coated with PA or LFn antigen by incubating 100 μL of a solution of 10 μg/nl antigen in 0.05 M sodium carbonate, pH 9.75 (Coating Buffer) overnight at room temperature (25° C.+5° C.). The plates were then washed once with Wash Buffer (PBS/0.05% Tween 20; Sigma Chemical Co., St. Louis, Mo., cat. #P1379). Assay Buffer was added to each well (300 μL/ well), and incubated at room temperature for 2 hours, for blocking. Assay buffer consisted of 1× Dulbecco's PBS (Life Technologies, Rockville, Md.; cat. #14200-075) with 0.5% aqueous cold water fish gelatin (Sigma Chemical Co., St. Louis, Mo., cat. #G7765), 0.6% Igepal C (Sigma, cat. #3021), 0.9% Triton X 100 (Sigma, cat. #T9284), 1% Protease-free BSA (Intergen, Purchase, N.Y., cat. #3100-01), 1% Blotting/Blocker Grade Non-fat Dry Milk (Bio-Rad Laboratories, Hercules, Calif., cat. #170-6404) and 1.0% ProClin 300 (Supelco, Bellefonte, PA, cat. #4-8127). The microtiter plate wells were aspirated, and the plates were patted dry on paper towels. The plates were allowed to air dry for at least 8 hours at 37° C., if they were not used immediately. If necessary, plates were stored with plate sealers in plastic bags at 4° C±2° C. for up to 1 month.

All serum samples were diluted in Assay Buffer, and 100 μL was put into each well, sealed, and incubated at room temperature for 2 hours or at 37° C. for 1 hour. Plates were washed 4 times with Wash Buffer, then patted dry on paper towels. Goat-anti-rabbit HRP reagent (peroxidase conjugated-AffiniPure Goat-anti-Rabbit IgG H+L, Jackson ImmunoResearch, West Grove, Pa., cat. #111-035-144) was diluted in Assay Buffer and was added at 100 μL/well, sealed, and incubated at room temperature for 2 hours or at 37° C. for 1 hour. Plates were washed 4 times with Wash Buffer, and then patted dry on paper towels. For detection, 100 μL/well of TMB (Sigma, cat. #T8665) was added and incubated at room temperature for 15 minutes, followed by 50 μL/well of Stop Solution (2 N H₂SO₄), and the O.D. was read at 450 nm.

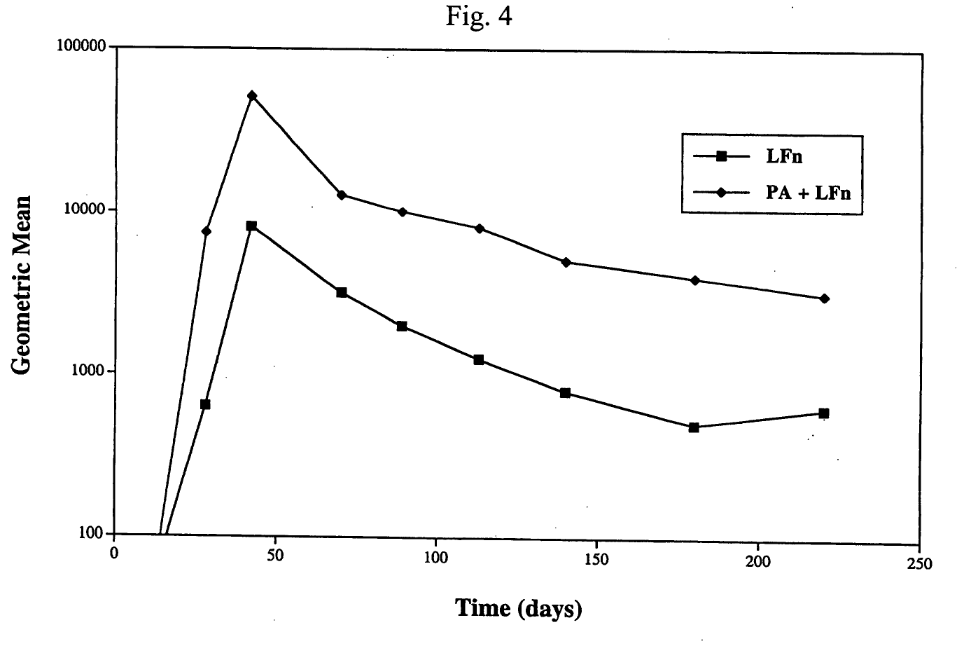

FIG. 3 shows the geometric mean anti-PA antibody titers of rabbits administered three injections of rPA and rPA+rLFn, respectively. The anti-PA titers resulting from both immunizations peaked at around 200,000 and was sustained above about 1000 even after 224 days, the time point where these results were plotted. FIG. 4 shows the geometric mean anti-LFn antibody titer of rabbits administered three injections of rLFn and rPA+rLFn, respectively. The anti-LFn titers resulting from immunization with rLFn alone peaked at around 10,000 and was sustained above 500 even after 224 days, the time point where these results were plotted. The anti-LFn titers resulting from immunization with a combination of rPA and rLFn peaked at around 50,000 and were sustained above 3000 even after 224 days, indicating that the inclusion of rPA adjuvanted the rLFn as an immunogen.

### EXAMPLE IV

Using procedures similar to Example III, the adjuvanting effect of rPA was tested in BALB/c mice using an immunogenic composition including rPA and LFn-OspA (i.e., a fusion protein comprised of LF amino acids 1-254 fused to another bacterial antigen, OspA (outer surface protein of Borellia burgdorferi).

Immunogenic compositions were formulated by dispersing the desired amount of rPA or LFn-OspA, or combinations thereof, in sterile saline. Dosage volumes were 0.1 mL. Two groups of five male BALB/c mice (Taconic, Germantown, N.Y.) were administered one of three immunogenic compositions in a series of three intramuscular (i.m.) injections, approximately two weeks apart (day 1, day 15, day 29). The parameters of the immunization are outlined in Table 3, below:

TABLE 3
Immunization with LFn-OspA or LFn-OspA + rPA

| | | rLFn- | | | |
|---|---|---|---|---|---|
| Compo- | rPA | OspA | Dose | Injection | |
| sition | Dose | Dose | vol. | Days | Blood Sample Days |
| LFn-OspA | 50 μg | 100 μg | 0.1 mL | 1, 15, 29 | 1*, 14, 28, 42, 70, 90, |
| and rPA | | | | | 115, 146, 183, 225, |
| | | | | | 267 |
| LFn-OspA | — | 100 μg | 0.1 mL | 1, 15, 29 | 1*, 14, 28, 42, 70, 90, |
| only | | | | | 115, 146, 183, 225, |
| | | | | | 267 |

*blood sample taken prior to initial injection

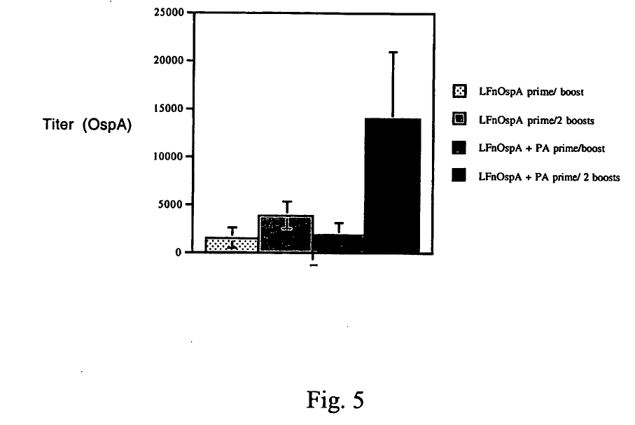

Anti-OspA titers were measured using the same type of ELISA as in Example III. Anti-OspA antibody titers were calculated from interim samples to show the effect of an initial injection plus one boost, compared with an initial injection followed by two boosts. The results are shown in FIG. 5. It can be seen that the anti-OspA titers for the two-component composition including both rPA and the LFn-OspA fusion protein showed a marked difference attributable to the inclusion of rPA after the second boost. This again indicates the adjuvanting effect of rPA.

### EXAMPLE V

### Assessment of the Biological Activity of Antisera From rPA-Immunized Rabbits

In order to determine the in vitro biological activity of antisera from the rabbits immunized with rPA in Example II, a toxin neutralization assay was performed. Such assays have become standardized and accepted as indicators of induction of protective immunity. See, e.g., Pittman et al., Vaccine, 20:1412-1420 (2002), and references cited therein.

The toxin neutralization assay is based on the fact that the combination of LF and PA is toxic to the macrophage cell line employed in the assay. Sera from an immunized subject is added to cells at differing dilutions in combination with lethal amounts of LF and PA. The viable cells remaining are measured using a reagent that is converted by live cells to a formazan that absorbs light at 490 nm.

Rabbit sera from Week 7 and Week 78 (see Table 1, supra) were selected as the likely timepoints for high titers based on previous EIA titer determination for Week 7 and the timing of the third boost (Week 74). A pool of sera from all rPA-immunized rabbits at each time point was prepared using equal volumes from all rabbits. These serum pools were titrated in the toxin neutralization assay and compared to normal rabbit serum.

Assay Method

The macrophage cell line, RAW 264.7, was grown from a vial obtained from the ATCC (TIB-71, RAW 264.7 Lot #1422325). These cells were maintained in Dulbecco's Modified Eagle's Medium supplemented with 10% heat inactivated fetal bovine serum and antibiotics (complete DMEM). Cells were passaged by scraping.

For the assay, 96-well flat bottomed plates were seeded with approximately 3×10⁴ cells/well in a volume of 100 μl of complete DMEM. Three wells were left empty for an assay blank. The plate was incubated for 3 days in a humidified CO₂ incubator at 37° C. Wells were visually inspected for confluence of the cells. Cells were >80% confluent.

Antisera pooled from Week 7 samples and Weed 78 samples were used and compared with normal rabbit serum (NRS). Serum samples were serially diluted by 1:10 in 96 U-bottomed plates from an initial dilution of 1:50. Complete DMEM was the diluent used. Recombinant PA (PA40) was adjusted to 4 μg/mL in complete DMEM. Recombinant LF (LF02) was adjusted to 2 μg/nl in complete DMEM. These concentrations are 80-fold and 50-fold greater, respectively, than the amounts of toxin components used in the validated assay of Pittman et al., supra.

To set up the assay, the culture medium was flicked out of the 96 well plate with the RAW 264.7 cells. Either 50 μl of the serum dilutions were added to the appropriate wells or 50 μl of medium. PA (25 μl) and LF (25 μl) were added to wells as appropriate or the same volume of medium. The controls were rPA only, rLF only, rPA and rLF in combination. The plate blank was the wells without cells plus rPA and rLF. The plate was incubated for 3 hours in a humidified CO₂ incubator at 37° C. After this incubation, 20 μl of Promega Cell Titer 96 Reagent (G-3580) was added to each well. The plates were incubated for an additional 2 hours and read at 490 nm in a microplate reader. Data were analyzed using SOFrmax Pro 3.1.2.

Both immune serum pools (Week 7, Week 78) inhibited the cell death induced by anthrax toxin (rPA+rLF). The data shown in Table 4 below clearly show that the effects of the antisera to rPA are easily demonstrated at titers of 1:1000. The combination of rPA and rLF reduces the viable cell value from around 2 to 0.2. The addition of immune serum inhibits this effect and produces values comparable to untreated cells at a 1:100 dilution (˜2.3) and only slightly below the untreated cell value at 1:1000 (˜1.8).

TABLE 4
Effect of anti-PA serum on cell death induced by
antrax toxin (PA + LF)

| serum dilution | NRS | Week 7 Serum | Week 78 Serum |
|---|---|---|---|
| 1/10² | 0.17 | 2.47 | 2.21 |
| 1/10³ | 0.26 | 1.89 | 1.77 |
| 1/10⁴ | 0.38 | 0.42 | 0.22 |
| 1/10⁵ | 0.25 | 0.32 | 0.30 |
| 1/10⁶ | 0.44 | 0.31 | 0.30 |
| 1/10⁷ | 0.13 | 0.14 | 0.37 |

controls:
PA only 2.25
LF only 2.00
PA + LF 0.21
Data shown are the average absorbance at 490 nm of at least triplicate wells minus the background

The titer for the 50% inhibition point for each immune serum pool was determined using a 4-parameter curve fit. This was a titer of 1:2273 for Week 7 and 1:1163 for Week 78.

Rabbit antisera reactive with PA inhibits the in vitro intoxication of macrophages produced by the toxin combination of rPA+rLF. Immunization with rPA thus generates an immune response that affords protection against anthrax toxin challenge, as determined in this biological assay. These results indicate that the rPA vaccinated rabbits would be protected against a wildtype anthrax challenge.

From the above description, effective immunogenic compositions for raising immune responses against anthrax antigens and effective methods for immunization against anthrax antigens can be readily prepared. By following the teachings above, the skilled practitioner will be able to prepare and practice the disclosed embodiments and many additional embodiments suggested by the foregoing disclosure. For example, substitution of polypeptide immunogens homologous to rPA and/or LFn as described herein to achieve the same or similar immune responses in mammalian subjects may be performed without departing from the teachings herein. Homologous rPA or LFn polypeptides having a segment of at least 10 amino acids having greater than 90% homology to the native PA or LFn amino acid sequence will be expected to elicit production of at least a subpopulation of the same anti-PA or anti-LFn antibodies as the immunogen having 100% sequence identity to the native PA or LFn sequence. As used and understood herein, “percent homology” or “percent identity” of two amino acid sequences or of two nucleic acid sequences is determined using the algorithm of Karlin and Altschul (Proc. Natl. Acad. Sci. USA, 87: 2264-2268 (1990)), modified as in Karlin and Altschul (Proc. Natl. Acad. Sci. USA, 90: 5873-5877 (1993)). Such an algorithm is incorporated into the NBLAST and XBLAST programs of Altschul et al. (J. Mol. Biol., 215: 403410 (1990)). BLAST nucleotide searches are performed with the NBLAST program to obtain nucleotide sequences homologous to a nucleic acid molecule described herein. BLAST protein searches are performed with the XBLAST program to obtain amino acid sequences homologous to a reference polypeptide. To obtain gapped alignments for comparison purposes, Gapped BLAST is utilized as described in Altschul et al. (Nucleic Acids Res., 25: 3389-3402 (1997)). When utilizing BLAST and Gapped BLAST programs, the default parameters of the respective programs (e.g., XBLAST and NBLAST) are used. See, www.ncbi.nlm.nih.gov. All such obvious variations in the teachings provided herein may be accomplished without undue experimentation or the application of inventive effort.

All of the publications cited herein are incorporated herein by reference in their entireties.

What is claimed is:

1. An immunogenic composition capable of raising an anti-B. anthracis antigen immune response in a mammal consisting essentially of recombinant B. anthracis Protective Antigen (rPA).

2. The immunogenic composition of claim 1 formulated without adjuvant.

3. An immunogenic composition capable of raising an anti-B. anthracis antigen immune response in a mammal consisting essentially of rPA and a truncated, non-toxic B. anthracis Lethal Factor (LFn).

4. The immunogenic composition of claim 3 formulated without adjuvant.

5. A method for eliciting an immune response in a mammalian subject against a B. anthracis antigen comprising:
(a) administering to a mammalian subject a composition consisting essentially of rPA,
(b) optionally, repeating said administration one or more times,
wherein said administration results-in an an anti-PA antibody response in said mammal.

6. The method of claim 5, wherein the amount of said rPA in each administration is greater than 50 μg rPA.

7. The method of claim 5, wherein the amount of said rPA in each administration is greater than 100 μg rPA.

8. The method of claim 5, wherein the amount of said rPA in each administration is greater than 250 μg rPA.

9. The method of claim 5, wherein the amount of said rPA in each administration is greater than 500 μg rPA.

10. The method of claim 5, wherein the amount of said rPA in each administration is greater than 1000 μg rPA.

11. The method of claim 5, wherein the composition is administered without using an adjuvant.

12. The method of claim 5, wherein the administration of rPA of step (a) is repeated three or fewer times.

13. The method of claim 11, wherein an anti-PA antibody titer exceeding 100 is achieved.

14. The method of claim 11, wherein an anti-PA antibody titer exceeding 1000 is achieved.

15. The method of claim 11, wherein an anti-PA antibody titer exceeding 10,000 is achieved.

16. The method of claim 11, wherein an anti-PA antibody titer exceeding 100,000 is achieved.

17. The method of claim 11, wherein said mammalian subject is immunized against subsequent B. anthracis infection.

18. A method of immunizing a mammalian subject against B. anthracis comprising:
(a) administering to a mammalian subject a composition consisting essentially of recombinant Protective Antigen (rPA),
(b) optionally, repeating said administration one or more times,
wherein said mammalian subject is thereby immunized against B. anthracis infection.

19. The method of claim 18, wherein the amount of said rPA in each administration is greater than 50 μg rPA.

20. The method of claim 18, wherein the amount of said rPA in each administration is greater than 100 μg rPA.

21. The method of claim 18, wherein the amount of said rPA in each administration is greater than 250 μg rPA.

22. The method of claim 18, wherein the amount of said rPA in each administration is greater than 500 μg rPA.

23. The method of claim 18, wherein the amount of said rPA in each administration is greater than 1000 μg rPA.

24. The method of claim 18, wherein the composition is administered without using an adjuvant.

25. The method of claim 18, wherein the administration of rPA of step (a) is repeated three or fewer times.

26. The method of claim 25, wherein an anti-PA antibody titer exceeding 100 is achieved.

27. The method of claim 25, wherein an anti-PA antibody titer exceeding 1000 is achieved.

28. The method of claim 25, wherein an anti-PA antibody titer exceeding 10,000 is achieved.

29. The method of claim 25, wherein an anti-PA antibody titer exceeding 100,000 is achieved.

30. A method for obtaining high-purity rPA which comprises:
(a) culturing recombinant bacterial host cells transformed to express recombinant Protective Anitgen (rPA),
(b) treating the cells to release the rPA into the culture medium,
(c) purifying the culture medium to isolate the rPA using a combination of purification steps comprising:
(i) anion exchange chromatography,
(ii) hydroxyapatite chromatography,
(iii) hydrophobic interaction chromatography, and
(iv) size exclusion chromatography.

31. The method of claim 30, wherein said host cells are E. coli cells.

32. The method of claim 30, wherein said purifying step (c) employs purification steps in the following order: (1) anion exchange chromatography, (2) hydroxyapatite chromatography, (3) hydrophobic interaction chromatography, and (4) size exclusion chromatography.

33. The method of claim 32, wherein the hydroxyapatite chromatography utilizes a ceramic hydroxyapatite matrix.

34. A method for obtaining high-purity recombinant LFn polypeptide which comprises:
(a) culturing bacterial host cells transformed to express recombinant LFn,
(b) lysing the host cells,
(c) purifying the host cell lysate using a combination of purification steps comprising:
(i) immobilized metal affinity chromatography,
(ii) anion exchange chromatography,
(iii) hydrophobic interaction chromatography, and
(iv) size exclusion chromatography.

35. The method of claim 34, wherein said host cells are E. coli cells.

36. The method of claim 34, wherein said purifying step (c) employs purification steps in the following order: (1) immobilized metal affinity chromatography, (2) anion exchange chromatography, (3) hydrophobic interaction chromatography, and (4) size exclusion chromatography.

37. A anthrax vaccination kit comprising:
(a) at least one container of an injectable solution of at least 50 μg of rPA,
(b) optionally, at least one container of an injectable solution of at least 50 μg of LFn,
(c) instructions for use of solution of rPA in according to the method of claim 18.

38. Use of pure recombinant PA in a vaccine regimen of up to four doses of 50 μg recombinant PA or more, to induce protective immunity to B. anthracis infection.
